SEQUENCE LISTING (1 sequence)

SEQ ID NO: 1 - protein, 140 aa, Homo sapiens
  alpha synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2019/0382346 A1
(43) Pub. Date: Dec. 19, 2019

(54) (NITRO-PHENYL)-NITROPYRIDINE COMPOUNDS FOR TREATING SYNUCLEINOPATHIES

(71) Applicants: UNIVERSITAT AUTONOMA DE BARCELONA, Barcelona (ES); UNIVERSIDAD DE ZARAGOZA, Zaragoza (ES)

(72) Inventors: Esther DALFO CAPELLA, Barcelona (ES); Salvador VENTURA ZAMORA, Barcelona (ES); Samuel PEÑA DÍAZ, Barcelona (ES); Jordi PUJOLS PUJOL, Barcelona (ES); Javier SANCHO SANZ, Zaragoza (ES); María CONDE GIMÉNEZ, Zaragoza (ES)

(73) Assignees: UNIVERSITAT AUTONOMA DE BARCELONA, Barcelona (ES); UNIVERSIDAD DE ZARAGOZA, Zaragoza (ES)

(21) Appl. No.: 16/488,128

(22) PCT Filed: Feb. 23, 2018
(86) PCT No.: PCT/EP2018/054540

§ 371 Date: Aug. 22, 2019

(30) Foreign Application Priority Data
Feb. 28, 2017 (EP) 17158468.3

(51) Int. Cl. C07D 213/84; A61P 25/28

(52) U.S. Cl. CPC C07D 213/84; A61P 25/28

(57) ABSTRACT

The present invention relates to compounds able to inhibit α-synuclein aggregation, their use in the treatment or prophylaxis of a synucleinopathy and to pharmaceutical compositions comprising said compounds.

The present invention relates to compounds able to inhibit α-synuclein aggregation, their use in the treatment or prophylaxis of a synucleinopathy and to pharmaceutical compositions comprising said compounds.

### BACKGROUND OF THE INVENTION

A number of neurodegenerative disorders, collectively referred to as α-synucleinopathies or simply synucleinopathies, are characterized by protein deposition in inclusions in neurons and/or glial cells, such as the so-called Lewy bodies and Lewy neurites, whose major component is α-synuclein.(Galvin, Lee and Trojanowski, 2001) Full-length α-synuclein is a 140 amino acid protein encoded by the SNCA gene. Alternative splice variants and single-point mutants are known. High concentrations of α-synuclein are found within neural tissues. α-synuclein can self-assemble so as to ultimately form insoluble aggregates. The localization of α-synuclein containing protein deposits directly correlates with the symptomatology observed in patients who suffer from a synucleinopathy. Disorders which are classified as synucleinopathies include Parkinson's Disease (PD), Dementia with Lewy Bodies (DLB), Multiple System Atrophy (MSA), Pure Autonomic Failure (PAF), Lewy Body Variant of Alzheimer's Disease (LBVAD) and Neurodegeneration with Brain Iron Accumulation (NBIA). See, e.g., (Benskey, Perez and Manfredsson, 2016) and the references cited therein.

(Herva et al., 2014) and (WO07110629 WISTA LABORATORIES LTD, 2007) describe several compounds as inhibitors of α-synuclein aggregation.

Parkinson's Disease is the second most common neurodegenerative disorder after Alzheimer's disease and is still incurable. It was therefore an object of the present invention to provide further compounds, which inhibit α-synuclein aggregation, and which can be used for the treatment or prophylaxis of Parkinson's Disease and other synucleinopathies.(Galvin, Lee and Trojanowski, 2001)

(WO2012080221 (UNIV LEUVEN KATH), 2012) discloses novel compounds for use in neurological disorders characterized by cytotoxic alpha-synculein.

(WO2010015816 (SUMMIT CORP PLC), 2010) discloses compounds for Lysosomal storage disorders and other proteostatic diseases including neurodegenerative diseases.

(WO2014014937 NEUROPORE THERAPEUTICS INC, 2014) discloses compounds inhibitors of protein aggregation for treating neurodegenerative disease including Parkinson Disease.

(US2010041747 (FISCHER GUNTER ET AL.), 2010) discloses compounds that inhibit isomerase activity

(EP0676397 SHIONOGI & CO, 2006) discloses compounds antagonistic for NMDA and AMDA receptors and are therapeutic agents for neurological disorders.

(US2004052822 (KOHARA TOSHUYUKI ET AL), 2004) discloses compounds that inhibit glycogen synthase kinase-3 for neurodegenerative disease such as Parkinson Disease (Moree et al., 2015) discloses the identification of a method to identify molecules that inhibit the aggregation of alpha-synuclein for the treatment of Parkinson Disease.

None of the mentioned references affects the present invention novelty but serve as prior references and state of the art for the present invention of therapeutic and preventive compounds for specifically neurodegenerative conditions.

### SUMMARY OF THE INVENTION

The present invention relates to a compound of formula I

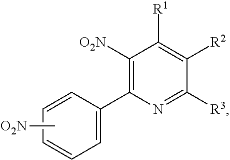

wherein

R¹ is selected from C₁-C₄-alkyl or cyclopropyl, wherein up to three hydrogen atoms of the C₁-C₄-alkyl or of the cyclopropyl are optionally substituted by radicals which are independently selected from F, Cl, OH and NH₂, provided there are no geminally bound OH groups if two or three OH groups are present,

R² is selected from −CN, Cl and F, and

R³ is selected from OH, C₁-C₄-alkoxy and C₁-C₄-alkylcarbonyloxy, or a tautomer thereof, a pharmaceutically acceptable solvate thereof, a prodrug thereof, or pharmaceutically acceptable salt thereof,

provided that the compound is not a compound of formula II

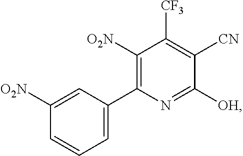 [SMILES: N#Cc1c(O)nc(-c2cccc([N+](=O)[O-])c2)c([N+](=O)[O-])c1C(F)(F)F]

formula IIb

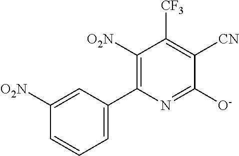 [SMILES: N#Cc1c([O-])nc(-c2cccc([N+](=O)[O-])c2)c([N+](=O)[O-])c1C(F)(F)F]

or formula III

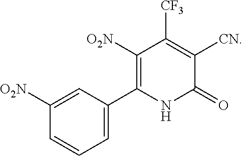 [SMILES: N#Cc1c(C(F)(F)F)c([N+](=O)[O-])c(-c2cccc([N+](=O)[O-])c2)[nH]c1=O]

The present invention further relates to a pharmaceutical composition comprising a compound of formula I

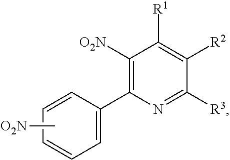

wherein

R¹ is selected from C₁-C₄-alkyl or cyclopropyl, wherein up to three hydrogen atoms of the C₁-C₄-alkyl or of the cyclopropyl are optionally substituted by radicals which are independently selected from F, Cl, OH and NH₂, provided there are no geminally bound OH groups if two or three OH groups are present,

R² is selected from —CN, CI and F, and

R³ is selected from OH, C₁-C₄-alkoxy and C₁-C₄-alkylcarbonyloxy,

or a pharmaceutically acceptable solvate thereof, a prodrug thereof, or a pharmaceutically acceptable salt thereof, and

at least one pharmaceutically acceptable carrier.

Provided the compound is not a compound of formula III

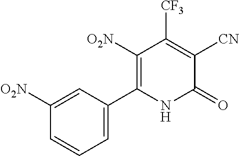 [SMILES: N#Cc1c(C(F)(F)F)c([N+](=O)[O-])c(-c2cccc([N+](=O)[O-])c2)[nH]c1=O]

The invention also relates to a compound of formula I, including the compounds of formulae II for use in medicine.

The invention further relates to a method for the treatment or the prophylaxis of a synucleinopathy in a subject, wherein a pharmaceutically effective amount of a compound of formula I, including the compounds of formulae II, IIb and III, is administered to the subject. For example, the invention relates to a method for delaying the onset or the progression of the synucleinopathy in the subject, wherein a pharmaceutically effective amount of a compound of formula I, including the compounds of formulae II, IIb and III, is administered to the subject.

The invention further relates to a compound of formula I, including the compounds of formulae II, IIb and III, for use in the treatment or prophylaxis of a synucleinopathy. For example, the invention relates to a compound of formula I, including the compounds of formulae II, IIb and III, for use in delaying the onset or the progression of the synucleinopathy.

The synucleinopathy may be selected from Parkinson's Disease, Dementia with Lewy Bodies, Multiple System Atrophy, Pure Autonomic Failure, Lewy Body Variant of Alzheimer's Disease and Neurodegeneration with Brain Iron Accumulation.

### BRIEF DESCRIPTION OF THE FIGURES

FIG. 1A shows α-synuclein aggregation kinetics performed on non-consecutive days. Thioflavin-T fluorescence was measured and serves as marker of α-synuclein aggregation. Error bars are represented as standard error. n=3.

FIG. 1B shows α-synuclein aggregation kinetics in the absence (“Control”) or the presence of Compound formula II, hereinafter mentioned as compound D (“D”, 2-hydroxy-5-nitro-6-(3-nitrophenyl)-4-(trifluoromethyl)nicotinonitrile). Thioflavin-T fluorescence was measured and serves as marker of α-synuclein aggregation. Error bars represent ±standard error. n=3.

FIG. 2 shows microscopic images of α-synuclein fibrils in the absence (“control”) or the presence of compound D.

FIG. 3 shows the inhibition of α-synuclein aggregation at different concentrations of compound D. Thioflavin-T fluorescence was measured and serves as marker of α-synuclein aggregation. Error bars represent±standard error. n=3. Statistics: ** p<0.005 and *** p<0.0005.

FIG. 4A shows human wild-type α-synuclein aggregation in the absence (“Control”) or the presence of compound D. Scattering of the samples was measured at 300 nm and 340 nm using a Varian fluorimeter and serves as marker of α-synuclein aggregation. Error bars represent±standard error. n=3. Statistics: ** p<0.005.

FIG. 4B shows aggregation of human H50Q mutant α-synuclein (“H50Q”) and human A30P mutant α-synuclein (“A30P”) in the absence (“Control”) or presence of compound D. Thioflavin-T fluorescence was measured and serves as marker of α-synuclein aggregation. Error bars represent±standard error. n=3. Statistics: *** p<0.0005.

FIG. 5A shows confocal images of C. elegans expressing α-synuclein fused to yellow fluorescent protein (YFP) in body wall muscle cells which were kept for 5 days in the absence (vehicle, DMSO) or the presence of compound D. White signal in all figures represents α-synuclein-YFP protein inclusions in muscle cells of the animals. Attached to each panel there is an expansion of each picture, delimited by the dashed square. Protein inclusions are labeled with white arrows.

FIG. 5B shows a quantification of the number of α-synuclein-YFP protein inclusions per area observed in the animals depicted in FIG. 5A. Error bars represent±standard error of mean (SEM). n=8. Statistics: Unpaired t-test. ** P=0.0079

FIG. 6 shows the results of a cytotoxicity analysis of compound D at concentrations in the range of 10-1000 μM. The analysis included reference samples with untreated cells (“control”) and cells treated only with the vehicle DMSO (“DMSO”). Fluorescence levels equal or higher than of the reference indicate absence of toxicity.

### DETAILED DESCRIPTION OF THE INVENTION

For the purposes of the present invention, the term “C₁-C₄-alkyl” refers to methyl, ethyl, n-propyl, isopropyl, n-butyl, isobutyl, sec-butyl or tert-butyl. Preferably, C₁-C₄-alkyl is selected from methyl, ethyl, n-propyl and isopropyl, in particular C₁-C₄-alkyl is methyl or ethyl, especially methyl.

The term “C₁-C₄-alkoxy” refers to methoxy, ethoxy, n-propoxy, isopropoxy, n-butoxy, tert-butoxy. Preferably, C₁-C₄-alkoxy is selected from methoxy, ethoxy and isopropoxy, more preferably from methoxy and ethoxy, in particular from methoxy.

The term “C₁-C₄-alkylcarbonyloxy” refers to a radical of formula —O—C(═O)—C₁-C₄-alkyl, wherein C₁-C₄-alkyl has one of the meanings given above. “C₁-C₄-alkylcarbonyloxy” is typically selected from methylcarbonyloxy, ethylcarbonyloxy, n-propylcarbonyloxy, isopropylcarbonyloxy, sec-butylcarbonyloxy, n-butylcarbonyloxy and tert-butylcarbonyloxy. Preferably, C₁-C₄-alkylcarbonyloxy is selected from methylcarbonyloxy, ethylcarbonyloxy, isopropylcarbonyloxy and tert-butylcarbonyloxy, more preferably from methylcarbonyloxy and ethylcarbonyloxy, in particular from methylcarbonyloxy.

Preferably, the radical R¹ in formula I is selected from C₁-C₄-alkyl and cyclopropyl, wherein up to three hydrogen atoms of the C₁-C₄-alkyl or of the cyclopropyl are optionally substituted by radicals which are independently selected from F and Cl.

More preferably, the radical R¹ in formula I is selected from methyl, ethyl and cyclopropyl wherein up to three hydrogen atoms of methyl, ethyl and cyclopropyl are optionally substituted by radicals which are independently selected from F and Cl.

Even more preferred, the radical R¹ in formula I is selected from methyl and cyclopropyl wherein up to three hydrogen atoms of methyl and cyclopropyl are substituted by radicals which are independently selected from F.

In particular, the radical R¹ in formula I is selected from methyl wherein up to three hydrogen atoms of methyl are substituted by radicals which are independently selected from F.

Especially, the radical R¹ in formula I is trifluoromethyl.

Preferably, the radical R² in formula I is selected from CN.

Preferably, the radical R³ in formula I is selected from OH and C₁-C₄-alkylcarbonyloxy.

More preferably, the radical R³ in formula I is selected from OH, methylcarbonyloxy and ethylcarbonyloxy.

Even more preferably, the radical R³ in formula I is selected from OH and methylcarbonyloxy.

In particular, the radical R³ in formula I is OH.

The preferred embodiments mentioned above may be combined arbitrarily with one another.

Accordingly, a preferred embodiment of the present invention relates to a compound of formula I

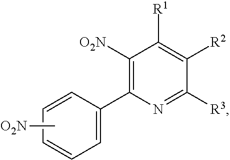

wherein

R¹ is selected from methyl, ethyl or cyclopropyl, wherein up to three hydrogen atoms of methyl, ethyl or of the cyclopropyl are optionally substituted by radicals which are independently selected from F and Cl,

R² is selected from —CN, Cl and F, and

R³ is selected from OH and methylcarbonyloxy,

or a tautomer thereof, a pharmaceutically acceptable solvate thereof, a prodrug thereof, or pharmaceutically acceptable salt thereof,

provided that the compound is not a compound of formula II

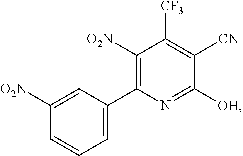 [SMILES: N#Cc1c(O)nc(-c2cccc([N+](=O)[O-])c2)c([N+](=O)[O-])c1C(F)(F)F]

formula IIb

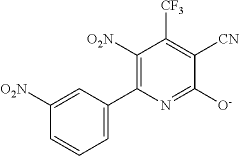 [SMILES: N#Cc1c([O-])nc(-c2cccc([N+](=O)[O-])c2)c([N+](=O)[O-])c1C(F)(F)F]

or formula III

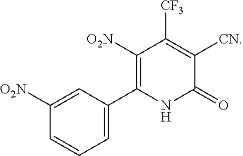 [SMILES: N#Cc1c(C(F)(F)F)c([N+](=O)[O-])c(-c2cccc([N+](=O)[O-])c2)[nH]c1=O]

An even more preferred embodiment of the present invention relates to a compound of formula I

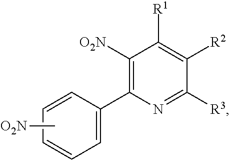

wherein

R¹ is selected from methyl or cyclopropyl, wherein up to three hydrogen atoms of methyl or of the cyclopropyl are substituted by F,

R² is —CN, and

R³ is selected from OH,

or a tautomer thereof, a pharmaceutically acceptable solvate thereof, a prodrug thereof, or pharmaceutically acceptable salt thereof,

provided that the compound is not a compound of formula II

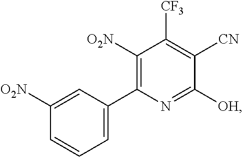 [SMILES: N#Cc1c(O)nc(-c2cccc([N+](=O)[O-])c2)c([N+](=O)[O-])c1C(F)(F)F]

formula IIb

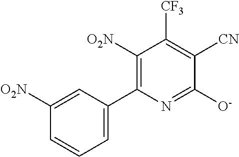 [SMILES: N#Cc1c([O-])nc(-c2cccc([N+](=O)[O-])c2)c([N+](=O)[O-])c1C(F)(F)F]

or formula III

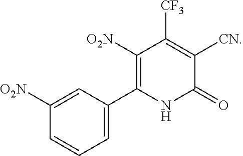 [SMILES: N#Cc1c(C(F)(F)F)c([N+](=O)[O-])c(-c2cccc([N+](=O)[O-])c2)[nH]c1=O]

A particular preferred embodiment of the present invention relates to a compound of formula I

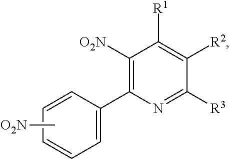

wherein

R¹ is trifluoromethyl,

R² is —CN, and

R³ is OH,

or a tautomer thereof, a pharmaceutically acceptable solvate thereof, a prodrug thereof, or pharmaceutically acceptable salt thereof,

provided that the compound is not a compound of formula II

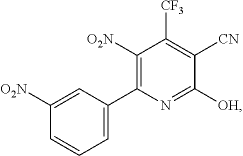 [SMILES: N#Cc1c(O)nc(-c2cccc([N+](=O)[O-])c2)c([N+](=O)[O-])c1C(F)(F)F]

formula IIb

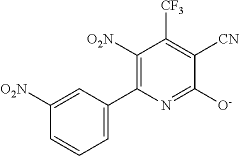 [SMILES: N#Cc1c([O-])nc(-c2cccc([N+](=O)[O-])c2)c([N+](=O)[O-])c1C(F)(F)F]

or formula III

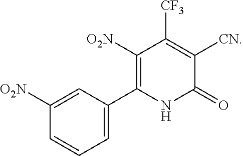 [SMILES: N#Cc1c(C(F)(F)F)c([N+](=O)[O-])c(-c2cccc([N+](=O)[O-])c2)[nH]c1=O]

Further preferred are compounds of the general formula I, wherein R³ is a hydroxyl group, i.e. compounds of the general formula Ia

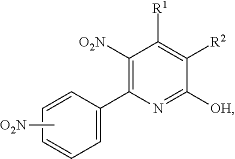

wherein

R¹ is selected from C₁-C₄-alkyl or cyclopropyl, wherein up to three hydrogen atoms of the C₁-C₄-alkyl or of the cyclopropyl are optionally substituted by radicals which are independently selected from F, Cl, OH and NH₂, provided there are no geminally bound OH groups if two or three OH groups are present, and

R² is selected from —CN, Cl and F,

or a tautomer thereof, a pharmaceutically acceptable solvate thereof, a prodrug thereof, or pharmaceutically acceptable salt thereof,

provided that the compound is not a compound of formula II

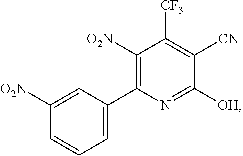 [SMILES: N#Cc1c(O)nc(-c2cccc([N+](=O)[O-])c2)c([N+](=O)[O-])c1C(F)(F)F]

formula IIb

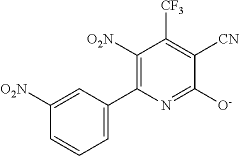 [SMILES: N#Cc1c([O-])nc(-c2cccc([N+](=O)[O-])c2)c([N+](=O)[O-])c1C(F)(F)F]

or formula III

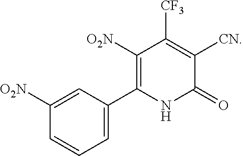 [SMILES: N#Cc1c(C(F)(F)F)c([N+](=O)[O-])c(-c2cccc([N+](=O)[O-])c2)[nH]c1=O]

Regarding the preferred and particularly preferred meanings of the radicals R¹ and R², reference is made to the statements given above.

The compounds of the general formula IA can also be present in form of their tautomers of the general formula Ib

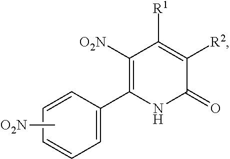

wherein R¹ and R² are as defined above.

Accordingly, the present invention also relates to compounds of formula Ib

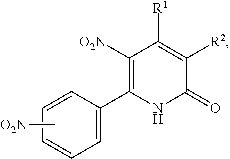

wherein

R¹ is selected from C₁-C₄-alkyl or cyclopropyl, wherein up to three hydrogen atoms of the C₁-C₄-alkyl or of the cyclopropyl are optionally substituted by radicals which are independently selected from F, Cl, OH and NH₂, provided there are no geminally bound OH groups if two or three OH groups are present, and

R² is selected from —CN, Cl and F,

or a pharmaceutically acceptable solvate thereof, a prodrug thereof, or a pharmaceutically acceptable salt thereof,

provided that the compound is not a compound of formula III

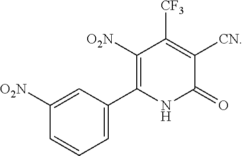 [SMILES: N#Cc1c(C(F)(F)F)c([N+](=O)[O-])c(-c2cccc([N+](=O)[O-])c2)[nH]c1=O]

Another embodiment of the present invention relates to a pharmaceutical composition comprising a compound of formula I

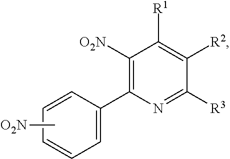

wherein

R¹ is selected from C₁-C₄-alkyl or cyclopropyl, wherein up to three hydrogen atoms of the C₁-C₄-alkyl or of the cyclopropyl are optionally substituted by radicals which are independently selected from F, Cl, OH and NH₂, provided there are no geminally bound OH groups if two or three OH groups are present,

R² is selected from —CN, Cl and F, and

R³ is selected from OH, C₁-C₄-alkoxy and C₁-C₄-alkylcarbonyloxy,

or a pharmaceutically acceptable solvate thereof, a prodrug thereof, or pharmaceutically acceptable salt thereof, and

at least one pharmaceutically acceptable carrier.

Provided the compound is not the compound of formula III

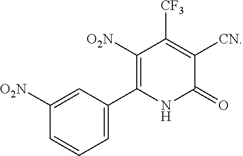 [SMILES: N#Cc1c(C(F)(F)F)c([N+](=O)[O-])c(-c2cccc([N+](=O)[O-])c2)[nH]c1=O]

Regarding the preferred and particularly preferred meanings of the radicals R¹, R² and R³, reference is made to the statements given above.

Accordingly, a preferred embodiment of the present invention relates to a pharmaceutical composition comprising a compound of formula I, wherein

R¹ is selected from methyl, ethyl or cyclopropyl, wherein up to three hydrogen atoms of methyl, ethyl or of the cyclopropyl are optionally substituted by radicals which are independently selected from F and Cl,

R² is selected from —CN, Cl and F, and

R³ is selected from OH and methylcarbonyloxy,

or a pharmaceutically acceptable solvate thereof, a prodrug thereof, or pharmaceutically acceptable salt thereof, and

at least one pharmaceutically acceptable carrier.

A more preferred embodiment of the present invention relates to a pharmaceutical composition comprising a compound of formula I, wherein

R¹ is selected from methyl or cyclopropyl, wherein up to three hydrogen atoms of methyl or of the cyclopropyl are substituted by F,

R² is —CN, and

R³ is OH,

or a pharmaceutically acceptable solvate thereof, a prodrug thereof, or pharmaceutically acceptable salt thereof, and

at least one pharmaceutically acceptable carrier.

An even more preferred embodiment of the present invention relates to a pharmaceutical composition comprising a compound of formula I, wherein

R¹ is trifluoromethyl,

R² is —CN, and

R³ is selected from OH and methylcarbonyloxy,

or a pharmaceutically acceptable solvate thereof, a prodrug thereof, or pharmaceutically acceptable salt thereof, and

at least one pharmaceutically acceptable carrier.

In particular, the present invention relates to a pharmaceutical composition comprising a compound of formula II

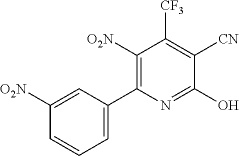 [SMILES: N#Cc1c(O)nc(-c2cccc([N+](=O)[O-])c2)c([N+](=O)[O-])c1C(F)(F)F]

or a pharmaceutically acceptable solvate thereof, a prodrug thereof, or pharmaceutically acceptable salt thereof.

Provided the compound is not the compound of formula III

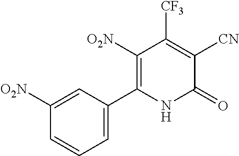 [SMILES: N#Cc1c(C(F)(F)F)c([N+](=O)[O-])c(-c2cccc([N+](=O)[O-])c2)[nH]c1=O]

A further preferred embodiment of the present invention relates to a pharmaceutical composition comprising a compound of formula la, as defined above, including the compound of formulae II,

or a pharmaceutically acceptable solvate thereof, a prodrug thereof, or pharmaceutically acceptable salt thereof, and

at least one pharmaceutically acceptable carrier.

A particularly preferred compound of formula I is the compound of formula II

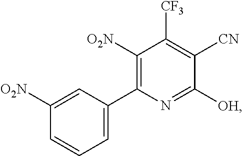 [SMILES: N#Cc1c(O)nc(-c2cccc([N+](=O)[O-])c2)c([N+](=O)[O-])c1C(F)(F)F]

2-hydroxy-5-nitro-6-(3-nitrophenyl)-4-(trifluoromethyl)nicotinonitrile (CAS Registry No. 685121-45-3). Database PubChem-NIH [Online]

U.S. National Library of Medicine; 30 May 2009 (2009-05-30), “ZINC08648827”,

XP002770781,

Database accession no. CID40782290

The compound of formula II may be present in its tautomeric form having formula III

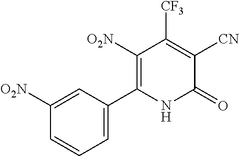 [SMILES: N#Cc1c(C(F)(F)F)c([N+](=O)[O-])c(-c2cccc([N+](=O)[O-])c2)[nH]c1=O]

5-nitro-6-(3-nitrophenyl)-2-oxo-4-(trifluoromethyl)nicotinonitrile, or as a mixture of the tautomers of formulae I and II.

Full-length human wild-type α-synuclein has the amino acid sequence set forth in SEQ ID NO:1.

(SEQ ID NO: 1)
MDVFMKGLSKAKEGVVAAAEKTKQGVAEAAGKTKEGVLYVGSKTKEG
VVHGVATVAEKTKEQVTNVGGAVVTGVTAVAQKTVEGAGSIAAATGF
VKKDQLGKNEEGAPQEGILEDMPVDPDNEAYEMPSEEGYQDYEPEA

Alternative splicing in exons 3 and 5 of the SNCA gene can result in α-synuclein isoforms having 126, 112 or 98 amino acids. See, e.g.,(Benskey, Perez and Manfredsson, 2016) and the references cited therein.

The compounds of formulae I and II, in particular the compound of formula II, can inhibit the in vitro and in vivo aggregation of α-synuclein, including splice variants and mutants thereof. Specifically, the compounds of formulae I and II can reduce said aggregation and/or the delay of the onset of said aggregation.

The compounds of formulae la and II may be used in the form of a prodrug thereof.

The term “prodrugs” means compounds which are metabolized in vivo to compounds of formula Ia and II. Typical examples of prodrugs are described in (Reinartz, Krafft and Hoyer, 2004) (Huttunen, Raunio and Rautio, 2011) and (Wermuth, 1996)C. These include for example phosphates, carbamates, amino acids, esters, amides, peptides, ureas and the like. As well as (Rautio et al., 2008) where it describes some of the common functional groups for prodrugs such as carbonates, esters, amides, carbamates, esthers, phosphates, oximes, imines, hydroxyl etc.

In particular embodiments, the compounds of the invention are present in the form of ester prodrugs thereof. The term “ester prodrugs” as used herein refers to esters formed between the hydroxy group of the compounds of formulae la and II and an acid. Suitable acids include, but are not limited to, amino acids, carboxylic acids and inorganic acids.

Expediently, the prodrug (e.g., the ester prodrug) is a metabolic precursor of the compounds of formulae la and II which is pharmaceutically acceptable.

Ideally, a prodrug is enzymatically stable in the blood, but is hydrolyzed so as to release the active parent compound as it reaches the target tissue. Esters are particularly suitable for the design of prodrugs for cerebral delivery due to the abundance of endogenous esterases in the CNS.

The prodrug may be inactive when administered to a subject but is metabolized in vivo to the compounds of formulae la and II or an active metabolite thereof. The term “active metabolite” as used herein refers to a metabolic product of the compounds of formulae Ia and II that is pharmaceutically effective, in particular a metabolic product that inhibits the aggregation of α-synuclein. Ester prodrugs of a known pharmaceutically active agent (drug) can be identified and generated using techniques well-known in the art. For review on the rational design of prodrugs see, e.g., (Huttunen, Raunio and Rautio, 2011).

According to one embodiment, the ester prodrug is an ester of the compounds of formulae la and II and an inorganic acid, wherein phosphate esters of the compounds of formula Ia and II are preferred.

According to another embodiment, the ester prodrug is an ester of the compounds of formulae Ia and II and an amino acid. The amino acid has a core structure containing an optionally alkylated amino group and a carboxyl group. The carbon atom attached to the carboxyl group is called the α-carbon. In α-amino acids both the amino and carboxyl group are attached to the α-carbon. In amino acids with a carbon side chain attached to the α-carbon, the carbons are labeled in the order of α, β, γ, δ, ϵ, etc. Amino acids with the amino group attached to a carbon other than the α-carbon are respectively called β-amino acids, γ-amino acids, δ-amino acids and so forth. α-amino acids can occur as D- or L-stereoisomers. Amino acid prodrug esters of the compounds of formula Ia and II include, but not limited to, proteinogenic amino acids and non-proteinogenic amino acids. Proteinogenic amino acids are α-amino acids and include canonical amino acids (arginine, histidine, lysine, aspartic acid, glutamic acid, serine, threonine, asparagine, glutamine, cysteine, glycine, proline, alanine, isoleucine, leucine, methionine, phenylalanine, tryptophan, tyrosine and valine) and non-canonical amino acids (e.g., selenocysteine, pyrrolysine), in particular the L-stereoisomers thereof. Examples of non-proteinogenic amino acids include ornithine, 3-aminopropanoic acid, homoarginine, citrulline, homocitrulline, homoserine, γ-aminobutyric acid, sarcosine, 2-aminoadipic acid, homocysteine, β-alanine, β-aminoisobutyric acid, γ-leucine, β-lysine, β-arginine, β-tyrosine, β-phenylalanine, isoserine, β-glutamic acid, β-tyrosine, β-dopa (3,4-dihydroxy-L- phenylalanine), 2-aminoisobutyric acid, isovaline, di-n-ethylglycine, N-methyl-alanine, 4-hydroxyproline, 5-hydroxylysine, 3-hydroxyleucine, 4-hydroxyisoleucine, 5-hydroxy-L-tryptophan, 1-aminocyclopropyl-1-carboxylic acid and azetidine-2-carboxylic acid.

According to another embodiment, the ester prodrug is an ester of the compounds of formulae Ia and II and a carboxylic acid. Suitable carboxylic acids include, for example, saturated, monounsaturated, polyunsaturated and acetylenic aliphatic carboxylic acids, including polycarboxylic acids. Examples of saturated carboxylic acids include, but are not limited to, methanoic, ethanoic, propanoic, butanoic, pentanoic, hexanoic, heptanoic, octanoic, 2-propylpentanoic acid, nonanoic, decanoic, dodecanoic, tetradecanoic, hexadecanoic, heptadecanoic, octadecanoic and eicosanoic acid. Examples of monounsaturated carboxylic acids include, but are not limited to, 4-decenoic, 9-decenoic, 5-lauroleic, 4-dodecenoic, 9-tetradecenoic, 5-tetradecenoic, 4-tetradecenoic, 9-hexadecenoic, 6-hexadecenoic, 6-octadecenoic and 9-octadecenoic acid. Examples of polyunsaturated carboxylic acids include, but are not limited to, sorbic, octadecadienoic, octadecatrienoic, octadecatetraenoic, eicosatrienoic, eicosatetraenoic, eicosapentaenoic, docosapentaenoic and docosahexaenoic acids. Examples of acetylenic carboxylic acids include, but are not limited to octadecynoic, octadecenynoic, 6,9-octadecenynoic, heptadecenynoic, tridecatetraenediynoic, tridecadienetriynoic, octadecadienediynoic, heptadecadienediynoic, octadecadienediynoic, octadecenediynoic and octadecenetriynoic acids. Examples of polycarboxylic acids include, but are not limited to, oxalic, malonic, succinic, glutaric, adipic, pimelic, suberic, sebacic, malic, tartaric, dihydroxymesoxalic, methylmalonic, fumaric, phthalic, isophthalic, terephthalic, citric and isocitric acids. Particular examples of useful carboxylic acids are fatty acids (e.g., stearic acid, linoleic acid, oleic acid).

Depending on the functionalities of the acids forming the ester prodrug with the compounds of formulae Ia and II, the ester prodrug can be neutral, or can comprise one or more acid or base functionalities which are able to form salts. The term “ester prodrug” as used herein includes compounds in the form of the free base or free acid as well as salts (in particular pharmaceutically acceptable salts) thereof. A pharmaceutically acceptable salt can be obtained, for example, by reacting an ester prodrug in the form of its free base with a suitable acid. Examples of suitable acids are known in the art and include, for example, hydrochloric acid, citric acid, tartaric acid, lactic acid, phosphoric acid, methanesulfonic acid, acetic acid, formic acid, maleic acid and fumaric acid.

The compounds of formulae I and II and prodrugs thereof may be present in the form of solvates, e.g. hydrates. As used herein, the term “solvates” designates crystalline forms of the compounds of formulae I and II, or a prodrug thereof, which comprise solvent molecules incorporated in the crystal lattice. The solvent molecules are preferably incorporated in stoichiometric ratios. Hydrates are a specific form of solvates; the solvent in this case is water.

What is described herein for the compounds of formulae I and II applies analogously to the tautomers, thereof, prodrugs thereof and solvates thereof. Thus, unless specified otherwise, in the methods, uses and pharmaceutical compositions described herein, the compounds of formulae I and II may be replaced by the tautomers thereof, an prodrug thereof, or a solvate thereof as described herein.

The present invention relates to pharmaceutical compositions comprising the compounds of formula I, including the compounds of formulae II and III, and at least one pharmaceutically acceptable carrier. The composition may optionally comprise one or more other therapeutic or prophylactic drugs for treating a synucleinopathy.

The term “pharmaceutically acceptable”, as used herein, refers to a compound that does not cause acute toxicity when administered in an amount that is required for medical treatment or medical prophylaxis. Expediently, all components of the pharmaceutical composition of the present invention are pharmaceutically acceptable.

Acceptable carriers can be a solid, semisolid or liquid material which serves as vehicle or medium for the pharmaceutically active compound. Pharmaceutically acceptable carriers are known in the art and are chosen according to the dosage form and the desired way of administration. For example, the composition can be formulated for oral, rectal, transdermal, subcutaneous, intravenous, intramuscular or intranasal administration.

The pharmaceutical compositions of the inventions can be, for example, solid dosage forms, such as powders, granules, tablets, in particular film tablets, lozenges, sachets, cachets, sugar-coated tablets, capsules, such as hard gelatin capsules and soft gelatin capsules, suppositories or vaginal medicinal forms, semisolid medicinal forms, such as ointments, creams, hydrogels, pastes or plasters, and also liquid medicinal forms, such as solutions, emulsions, in particular oil-in-water emulsions, suspensions, for example lotions, injection preparations and infusion preparations, and eyedrops and eardrops. Implanted release devices can also be used for administering inhibitors according to the invention. In addition, it is also possible to use liposomes or microspheres.

Suitable carriers are listed in the specialist medicinal monographs. In addition, the compositions can comprise pharmaceutically acceptable auxiliary substances, such as wetting agents; emulsifying and suspending agents; preservatives; antioxidants; anti-irritants; chelating agents; coating auxiliaries; emulsion stabilizers; film formers; gel formers; odor masking agents; taste corrigents; resin; hydrocolloids; solvents; solubilizers; neutralizing agents; diffusion accelerators; pigments; quaternary ammonium compounds; refatting and overfatting agents; raw materials for ointments, creams or oils; silicone derivatives; spreading auxiliaries; stabilizers; sterilants; suppository bases; tablet auxiliaries, such as binders, fillers, glidants, disintegrants or coatings; propellants; drying agents; opacifiers; thickeners; waxes; plasticizers and white mineral oils. Such auxiliary substances are also well known in the art.

The compounds of formula I, including the compounds of formulae II and III, can be used for the treatment or the prophylaxis of a synucleinopathy.

Synucleinopathies are a group of disorders characterized by protein deposition in inclusions located in neuronal and/or glial cells. Said protein deposits are referred to as Lewy bodies and Lewy neurites. The major component of said protein deposits is α-synuclein. The α-synuclein aggregation observed in these disorders is believed to be responsible for the neurotoxicity underlying their pathology. Various animal models have been developed to study the formation α-synuclein-containing protein deposits and their pathology in synucleinopathies. See, e.g.,(Benskey, Perez and Manfredsson, 2016) and the references cited therein.

Administration of the compounds of formula I, including the compounds of formulae II and III, can prevent and/or delay the onset or the progression of the formation of α-synuclein deposits in a subject, e.g. a subject known or suspected to have or being at risk of developing a synucleinopathy.

The treatment of a synucleinopathy as described herein can comprise one or more of the following: reducing or ameliorating the severity and/or duration of the synucleinopathy or one or more symptoms thereof, preventing the advancement of the synucleinopathy, causing regression of the synucleinopathy, preventing or delaying the recurrence, development, onset or progression of the synucleinopathy or one or more symptoms thereof, enhancing or improving the therapeutic effect(s) of another therapy (e.g., another therapeutic drug) against the synucleinopathy. Unless indicated otherwise, a treatment of a synucleinopathy as described herein may be a prophylactic treatment, e.g. in a subject at risk of developing a synucleinopathy. Prophylaxis or a prophylactic treatment of a synucleinopathy as described herein can include one or more of the following: preventing or delaying the onset of the synucleinopathy or one or more symptoms thereof, enhancing or improving the prophylactic effect of another therapy (e.g., another prophylactic drug) against the synucleinopathy.

The subject of the treatment or the prophylaxis according to the present invention can be a mammal and is preferably a human. The subject is expediently an individual known or suspected to suffer from a synucleinopathy, or at risk of developing a synucleinopathy.

Whether treatment or prophylaxis of a synucleinopathy using a compound of formula I is indicated, and in which form it is to take place, depends on the individual case and is subject to medical assessment (diagnosis) which takes into consideration signs, symptoms and/or malfunctions which are present, the risks of developing particular signs, symptoms and/or malfunctions, and other factors.

As a rule, treatment or prophylaxis is effected by means of single or repeated administration of a pharmaceutically effective amount of a compound of formula I, where appropriate together, or alternating, with other drugs or drug-containing compositions. As used herein, the term “pharmaceutically effective amount” refers to the amount of a therapy which is sufficient to achieve one or more of the following: reduce or ameliorate the severity and/or duration of the disease or one or more symptoms thereof, prevent the advancement of the disease, cause regression of the disease, prevent or delay the recurrence, development, onset or progression of the disease or one or more symptoms thereof, enhance or improve the therapeutic effect(s) of another therapy or prophylaxis (e.g., another therapeutic or prophylactic drug) against the disease.

The compounds of formula I, including the compounds of formulae II and III, can be administered in the form of a pharmaceutical composition of the invention. The formulation of the composition is expediently chosen according to the intended way of administration. Suitable formulation types for the different ways of administration are known in the art and described herein.

Examples of synucleinopathies which can be treated, delayed or prevented as described herein include Parkinson's Disease, Dementia with Lewy Bodies, Multiple System Atrophy, Pure Autonomic Failure, Lewy Body Variant of Alzheimer's Disease and Neurodegeneration with Brain Iron Accumulation. According to a particular embodiment, the synucleinopathy to be treated, delayed or prevented as described herein is Parkinson's Disease.

In Parkinson's Disease, Dementia with Lewy Bodies, Pure Autonomic Failure and Lewy Body Variant of Alzheimer's Disease, the α-synuclein-containing protein deposits are primarily detected in neurons. In Multiple System Atrophy, the deposits are primarily in glial cells. In Neurodegeneration with Brain Iron Accumulation α-synuclein-containing protein deposits are detected in both neurons and glial cells.

Certain point mutations of human α-synuclein are known in the art to significantly increase oligomerization. For example, the point mutations A30P, E46K, G51D, A53E and A53T of α-synuclein are known to cause familial forms of Parkinson's Disease. The compounds of formulae I, including the compounds of formulae II and III, also inhibits aggregation of such α-synuclein point mutants. Parkinson's Disease is a progressive disease which usually manifests after the age of 50 years, although early-onset cases (before 50 years) are known. The majority of the cases are sporadic suggesting a multifactorial etiology based on environmental and genetic factors. However, in some cases, there is a positive family history for the disease. Such familial forms of the Parkinson's Disease usually begin at an earlier age. See (I.F. et al., 2015).

In a particular embodiment, the subject to be treated according to the present invention suffers from a familial form of a synucleinopathy, for example from familial Parkinson's Disease. The subject suffering from a familial form of a synucleinopathy may comprise α-synuclein having at least one amino acid substitution selected from A30P, E46K, G51 D, A53E and A53T (amino acid positions numbered relative to full length α-synuclein as set forth in SEQ ID NO:1).

The compound of formula II can be also obtained from Aurora Screening Library, Aurora Fine Chemicals LLC 7929 Silverton Ave. Suite 609 San Diego, Calif., 92126, United States.

The compound of formula II can be prepared according to the scheme below. Reactions a), b) and c) of the scheme can be performed analogously to those described in JP 2004-026652A. Reactions d) and e) of the scheme can be performed analogously to those described in ES 380931A. And reaction f) of the scheme can be performed analogously to that described in (Howard et al., 2015)and (Miyaura and Suzuki, 1995).

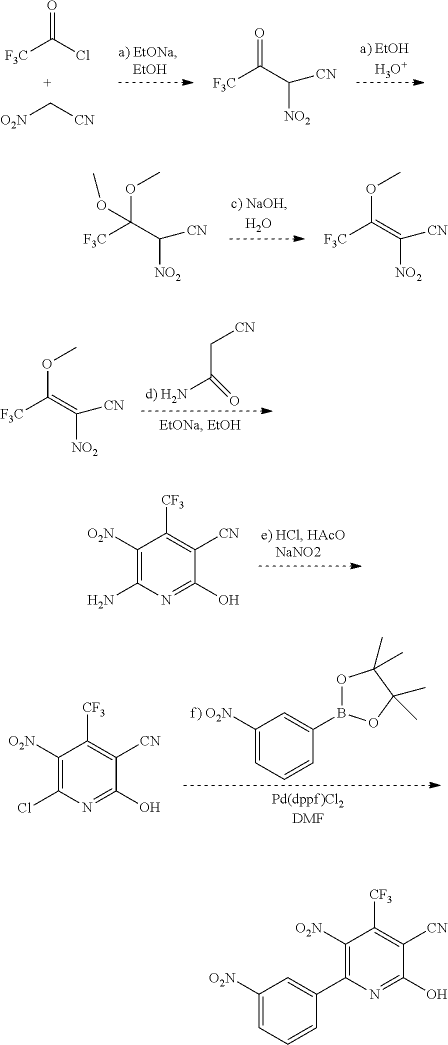

The synthesis sequence starts with the acylation reaction of the nitroacetonitrile 2 to afford the trifluoromethane compound 3 which will be submitted to ketalization and subsequent elimination to deliver the olefin 5. The 5-amino pyridine 7 could be obtained by ring formation, which would be converted to the desired intermediate 8 through a diazonium salt. Finally, the desired compound 10 could be prepared by using the palladium catalyzed Suzuki cross-coupling reaction of commercially available 3-nitrophenylboronic acid 9 with the 5-chloropyridine 8.

The compounds of the general formula I can be prepared using a similar reaction sequence.

The invention is explained in more detail below by means of examples. However, the examples are not to be understood to limit the invention in any way.

### EXAMPLES

### Methods

1. Expression and Purification of Human α-synuclein

Human α-synuclein was expressed and purified adapting a previous protocol from Volles and Lansbury (J Mol Biol 2007, 366:1510-1522). E. coli BL21 (DE3) cells were transformed with a pET21a plasmid (Novagen) containing the α-synuclein cDNA, grown in LB medium containing 100 μM/mL ampicillin and induced with 1 mM IPTG for 4 hours at an optical density at 600 nm of 0.6. After cell centrifugation at 7000×g for 10 min at 4° C., the pellet was resuspended in 20 mL Phosphate Buffered Saline (PBS) buffer, centrifuged again at 4000×g for 20 min at 4° C. and frozen at −80° C. When needed, the pellets were defrosted and resuspended in 10 mL lysis buffer (50 mM Tris pH 8, 150 mM NaCl, 1 μg/mL pepstatin, 20 μg/mL aprotinin, 1 mM benzamidine, 1 mM PMSF, 1 mM EDTA and 0.25 mg/mL lysozyme) prior to sonication using a LabSonic®U sonicator (B. Braun Biotech International) with a power level of 40 W and a repeating duty cycle of 0.7 sec for 3 intervals of 3 min. Resultant cell extract was boiled at 95° C. for 10 min and centrifuged at 20000×g for 40 min at 4° C. To the obtained supernatant 136 μL/mL of 10% w/v streptomycin sulfate and 228 μL/mL of pure acid acetic were added and centrifuged at 4° C. (20000×g, 10 min). The resulting soluble fraction was diluted with saturated ammonium sulfate (550 g/l) 1:1 (v/v) and centrifuged at 4° C. (20000×g, 10 min). Then, the pellet was resuspended in 50% ammonium sulfate and centrifuged at 4° C. (20000×g, 10 min). The pellet was washed with 100 mM pH 8 ammonium acetate (5 mL per culture litre) and pure EtOH 1:1 (v/v), then, the mixture was centrifuged at 4° C. (20000×g, 10 min). The pellet was resuspended in 20 mM pH 8 Tris and filtered with a 0.45 mm filter. Anion exchange column HiTrap Q HP was coupled to an ÄKTA purifier high performance liquid chromatography system in order to purify α-synuclein. Tris 20 mM pH 8 and Tris 20 mM pH 8, NaCl 1 M were used as buffer A and buffer B respectively. After column equilibration with buffer A, the sample was injected by using a Pump Direct Loading P-960 and the weak bonded proteins were washed with 5 column volumes (cv) of Buffer A. To properly isolate α-synuclein, a step gradient was applied as follows: i) 0-20% buffer B, 5 cv; ii) 20-45% buffer B, 11 cv; iii) 100% buffer B, 5 cv, obtaining pure α-synuclein between 25-35% buffer B concentration. The collected peaks were dialyzed in 5 L ammonium sulfate 50 mM overnight. α-synuclein concentration was determined measuring the absorbance at 280 nm and using the extinction coefficient 5960 M⁻¹ cm⁻¹. Purity was checked using 15% SDS-PAGE and unstained Protein Standard markers from Thermo Fisher Scientific. The gel was stained with comassie brilliant blue. Identity was checked by mass spectrometry. 2 μL of protein were dialysed for 30 minutes at room temperature using 20 mL of 50 mM ammonic bicarbonate and a 0.025 μm pore membrane (Millipore). After that, MALDI-TOF was analysis was performed with a ground steel plate and 2,6-dihidroxiacetophenone acid as a matrix, in a MALDI-TOF UltrafleXtreme (Bruker Daltonics). A 1:1 sample:matrix mixture was used, adding just 1 μL of these sample to the plate. For the analysis, a lineal mode was used with an accelerated voltage of 25 kV. Finally, after lyophilization, the protein was kept at −80° C.

2. Quenching Analysis

The tested compound was dissolved at 50 mM in DMSO. In order to check for interference of the compound with thioflavin-T (Th-T) excitation or emission, the absorption spectrum was measured at a concentration of 100 μM in 1×PBS and within a range of from 400 to 600 nm using a spectrophotometer Cary100.

3. α-synuclein Aggregation and Thioflavin-T Assays

Previously lyophilized α-synuclein was carefully dissolved in PBS buffer to a final concentration of 210 μM and filtered through a Millipore s 0.22-μm filter. α-synuclein aggregation assay was performed in a 96 wells plate (non-treated, black plastic) containing in each well a Teflon polyball (3.175 mm in diameter), 40 μM thioflavin-T, 70 μM α-synuclein, 100 μM of the tested compound and PBS up to a final volume of 150 μL. Plates were fixed into an orbital culture shaker Max-Q 4000 Thermo Scientific to keep the incubation at 37° C., 100 rpm. Every 2 hours, the fluorescence intensity was measured using a Victor3.0 Multilabel Reader by exciting the mixtures with 430-450 filter and collecting the emission intensity with 480-510 filter (triplicates for each measurement). Each plate contained 3 α-synuclein controls in the absence of any compound. The averaged Th-T fluorescence obtained for these wells at the end of the experiment was normalized to 1 and the kinetic curves in the different wells re-scaled accordingly. Re-scaled curves were used to compare the controls with the effect of the tested compound and to ensure that the controls were reproducible between different experiments. For the titration assay, different concentrations of tested compound (200, 150, 100, 75 and 25 μM) were used. For the scattering assay, 70 μM α-synuclein, 100 μM of the tested compound or DMSO (in control samples) and PBS up to a final volume of 1 mL were incubated in low-binding plastic tubes (Protein LoBind Tube 1.5 mL, Eppendorf) using a thermomixer (Thermomixer comfort, Eppendorf) at 37° C. and 600 rpm. After 2 weeks, scattering of the samples was analyzed at 300 nm and 340 nm using a Varian fluorimeter.

Each sample was tested in triplicate. Each plate contained an also triplicated control without tested compound.

4. Transmission Electron Microscopy (TEM)

α-synuclein fibers from final point reaction (either in absence or presence of the final concentration inhibitors) were collected in Eppendorfs. After diluting the aggregated α-synuclein to a concentration of 10 μM α-synuclein, each sample was sonicated for 10 minutes. 5 μL of these samples were placed on carbon-coated copper grids and allowed to stand for 5 minutes. Then, samples were carefully dried with filter paper to remove the excess of sample. Grids were washed twice with MiliQ water by immersion and stained by incubating grids with 5 μL 2% (w/v) uranyl acetate for 2 minutes for the negative staining. After removal of the uranyl acetate excess with filter paper, grids were let to air-dry for 10 minutes. The samples were imaged using a Jeol 1400 Transmission Electron Microscopy operating at an accelerating voltage of 120 kV. 30 fields were screened at least, to obtain representative images.

5. Statistical Analysis

Data were analyzed by ANOVA Tukey test using SPSS software. All data are shown as means and standard error. p<0.05 was considered statistically significant and indicated by ** and *** if p<0.005 and p<0.0005, respectively.

6. C. elegans α-synuclein Aggregation Model

α-synuclein aggregation was assessed using an C. elegans in vivo model described by (Van Ham et al., 2008) and (Muñoz-Lobato et al., 2014).

The nematode strain NL5901, unc-119(ed3) III; [pkls2386 (Punc-54::α-syn::yfp; unc-119(+))] was obtained from the Caenorhabditis elegans Genetic Center (CGC), University of Minnesota, USA. The strain expresses a fusion of human wildtype α-synuclein and yellow fluorescent protein (YFP) in body wall muscle cells. The nematodes were maintained using standard procedures, grown in NGM agar plates and fed with E coli (OP50 strain). Adult worms were bleached to get synchronized nematode cultures. NGM-agar plates with a) DMSO only (vehicle) and b) 10 μM final concentration of the tested compound were prepared. Afterwards, OP50 containing a) or b) was added to NGM plates and let dry for 24 h. Plates were stored at 4° C. and covered with aluminum foil until the day of the experiment. The next day, synchronized worms at L4 stage of development were added to the plates. Worms were passed to new plates every 48 h. After 5 days of development (L4+5) the numbers of α-synuclein aggregates were determined using a fluorescence microscope. To this end, the worms were washed from the plates with M9 buffer and added to glass slides containing 6% agarose and 100 mM sodium azide as anesthetic. The slides were covered with a coverslip and examined using 20× and 40× objectives. The same section in each animal was analyzed and captured in stacks to include aggregates contained from the top to the bottom of each animal (1 μM, 25 stacks). Image analysis was performed using ImageJ software, from the Z MAX acquisition, quantifying the number of α-synuclein-YFP aggregates. Final quantification and statistics was performed by the Graph Pad Prism 6.0 software, comparing vehicle-treated worms with drug-treated animals.

### Example 1: Effect on In Vitro Aggregation Kinetics of α-synuclein

In the aggregation assay described above, α-synuclein aggregates within approximately 24 h with a sigmoidal aggregation curve. The aggregation progress was tracked by monitoring the fluorescence of the amyloid specific reporter thioflavin-T. False positive results caused by thioflavin-T fluorescence quenching during data collection were excluded by a quenching analysis as described above which confirmed that 2-hydroxy-5-nitro-6-(3-nitrophenyl)-4-(trifluoromethyl)nicotinonitrile (“Compound formula II, herein after mentioned as compound D”) did not absorb at the thioflavin-T excitation or emission wavelengths, 450 and 480 nm, respectively.

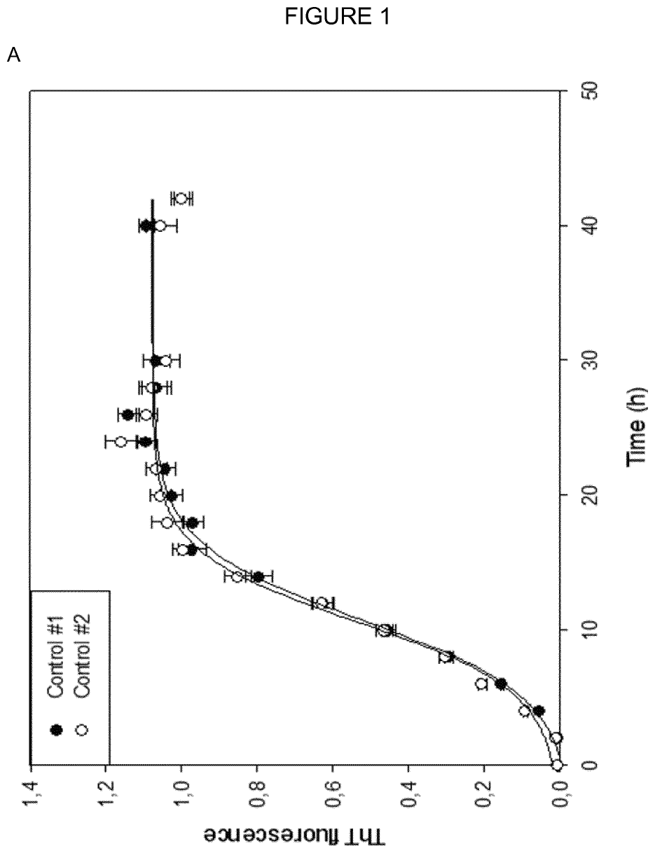

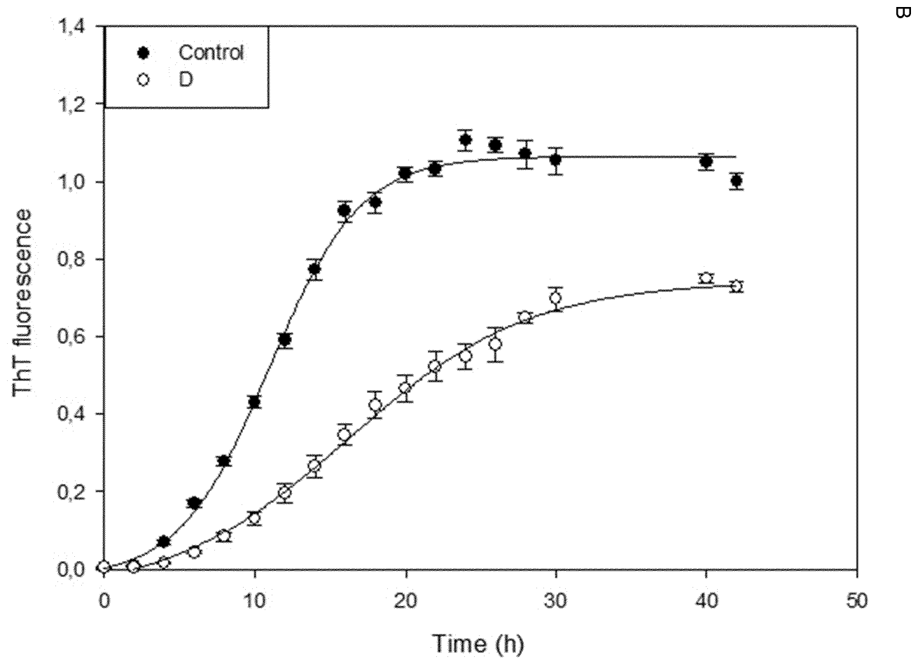

Compound D showed significant inhibition of human wild-type α-synuclein aggregation observed as thioflavin-T fluorescence (FIG. 1B). Specifically, compound D showed 32.4% inhibition at the end of the aggregation reaction relative to the control, wherein the halftime of aggregation was 3 h delayed relative to that of the control.

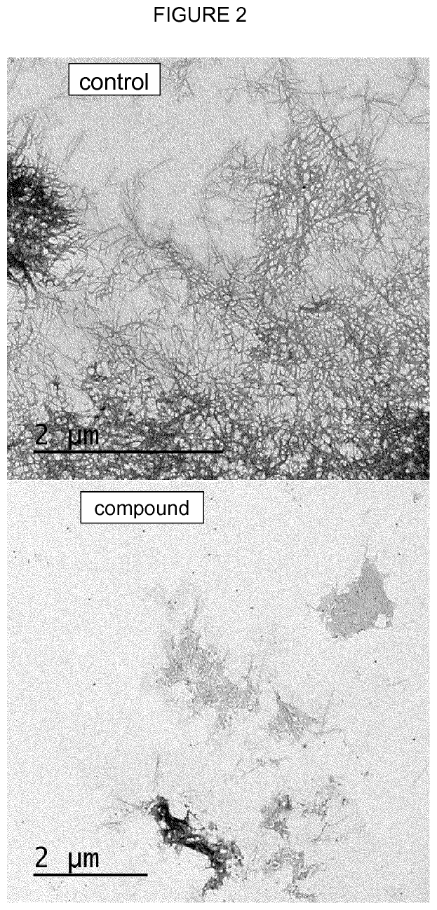

As can be seen by the very small error bars and the comparison of control aggregation curves measured on non-consecutive days (FIG. 1A), the results were of high statistical significance. Fitting the experimental data to the Finke-Watzky curve resulted in correlation coefficients of R>0.985. The results were confirmed by visual analysis of the samples via Transmission Electron Microscopy at the end of the aggregation reaction, wherein samples comprising compound D showed a significant decrease in both number and size of α-synuclein fibrils compared to the control samples (FIG. 2).

Aggregation curves were fitted and k1 (nucleation rate constant) and k2 (growth rate constant) were calculated using the Finke-Watzky two-step model (Watzky et al., 2008). Compound D decreased the final (i.e., reaction end point) thioflavin-T fluorescence as well as the kinetic constants k1 and k2.

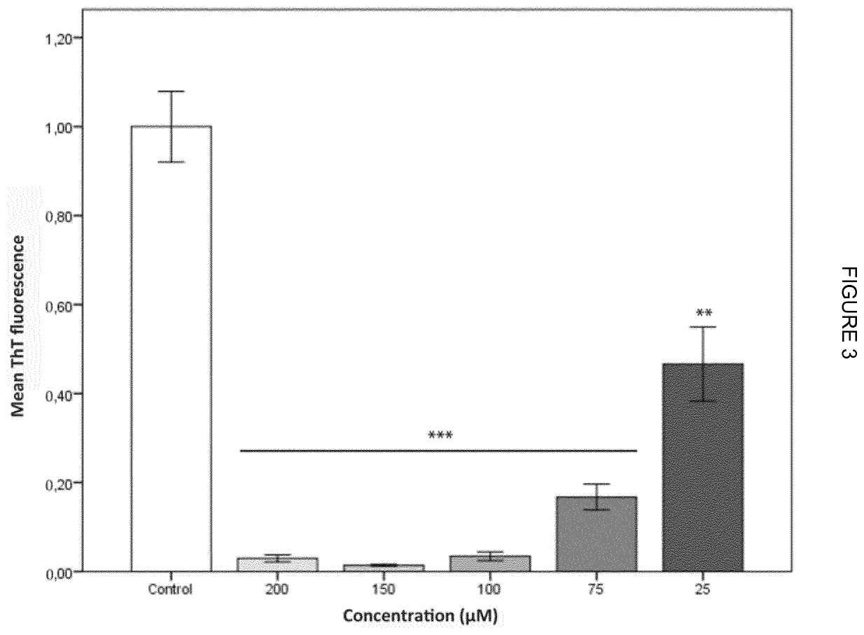

These results indicate that compound D delayed the onset of the aggregation reaction. Moreover, this compound had a clear dose-dependent anti-aggregation activity in titration assays and showed activity even at sub-stoichiometric protein:compound ratios (FIG. 3).

### Example 2: Effect on In Vitro Aggregation of Wild-type and Mutant α-synuclein

Human wild-type α-synuclein, human H50Q mutant α-synuclein and human A3OP mutant α-synuclein were prepared, lyophilized and dissolved in PBS using the methods described above.

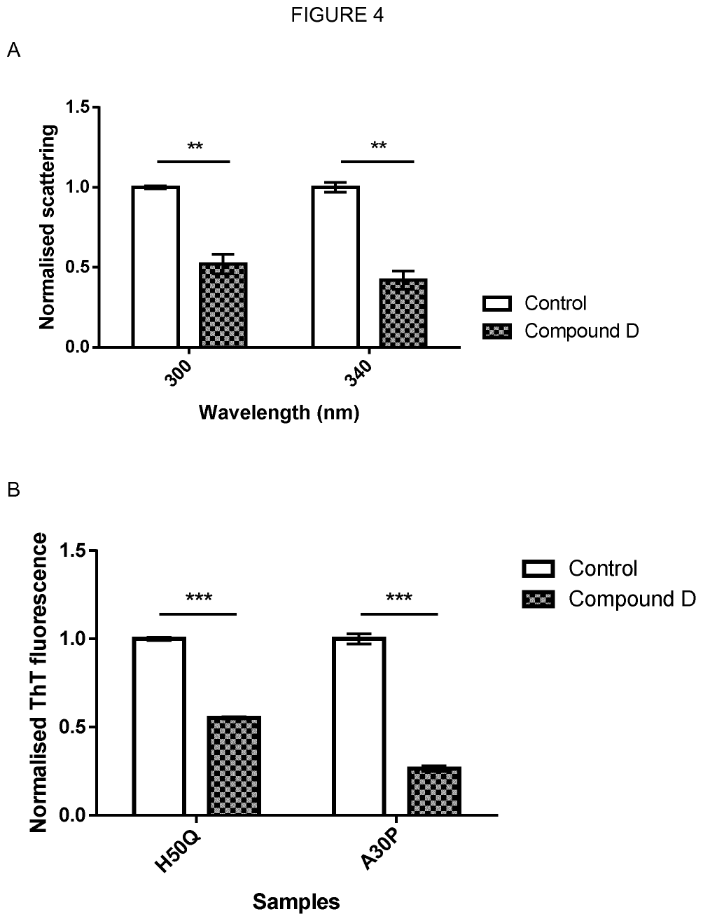

Human wild-type α-synuclein was incubated in the absence (control) or the presence of compound D. Conditions: triplicated samples, non-treated, low-binding plastic tubes (Protein LoBind Tube 1.5 mL, Eppendorf), 70 μM α-synuclein, 100 μM compound D, PBS up to a final volume of 1 mL, shaking on an thermomixer (Thermomixer comfort, Eppendorf) at 600 rpm and at 37° C. After 2 weeks, scattering of the samples was analyzed at 300 nm and 340 nm using a Varian fluorimeter. The results are shown in FIG. 4A.

Human H50Q mutant α-synuclein or human A30P mutant α-synuclein was incubated in the absence (control) or presence of compound D. Conditions: triplicated samples, non-treated, black plastic 96 wells plate containing a Teflon polyball (3.175 mm in diameter) in each well, 40 μM thioflavin-T, 70 μM α-synuclein, 100 μM compound D, PBS up to a final volume of 150 μL per well, shaking on an orbital culture shaker (Max-Q 4000 Thermo Scientific) at 100 rpm and at 37° C. Every 2 hours, the thioflavin-T fluorescence intensity was measured as described above. FIG. 4B shows normalized thioflavin-T fluorescence values at 24 h, when maximum fluorescence was observed.

The results confirm that compound D inhibits human wild-type α-synuclein as well as human H50Q mutant α-synuclein and human A3OP mutant α-synuclein.

### Example 3: Effect in a C. elegans α-synuclein Aggregation Model

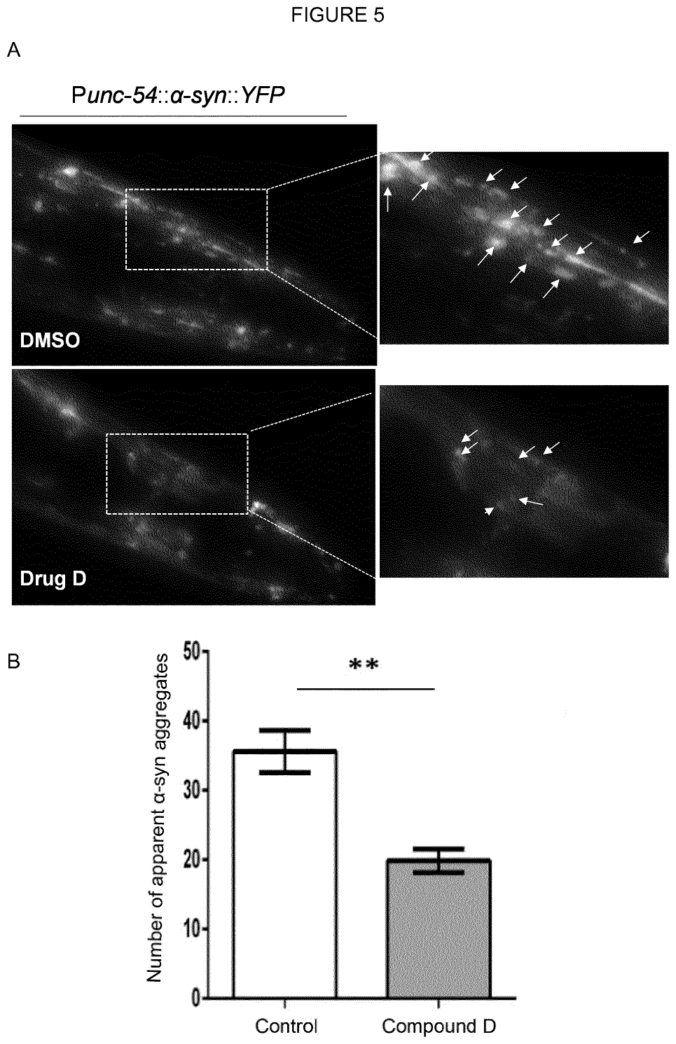

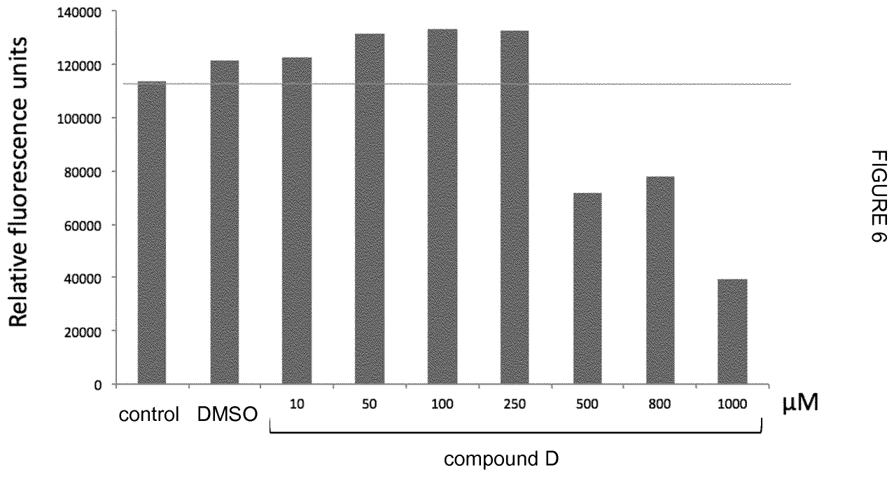

The effect of compound D was tested in a C. elegans in vivo aggregation model expressing human wild-type α-synuclein fused to yellow fluorescent protein (YFP) in the body wall muscle cells using the method described above. Worms at the L4 stage of development were incubated in the absence (control) or the presence of compound D. Representative confocal images obtained from the animals after 5 days of incubation show fluorescence representing protein inclusions comprising α-synuclein-YFP in the muscle cells of the worms. Treatment with compound D significantly decreased said protein inclusions (FIGS. 5A-B).

### Example 4: Cytotoxicity Assay

Cultured cells of human neuroblastoma cell line SH-SY5Y were incubated for 72 h with either the vehicle (DMSO) or with different concentrations of the compound. Then, the cells were incubated with a cell viability indicator (PrestoBlue® Cell Viability Reagent). The modification of PrestoBlue® Cell Viability Reagent by the reducing environment of viable cells turns the dye red in color becoming highly fluorescent. This color change can be detected measuring fluorescence by exciting at 531 nm and detecting emission at 615 nm. The CC50 of compound D (concentration that caused death in 50% of the cells, i.e. reduction to 50% viability) was >700 μM (FIG. 6). This indicates that compound D does not exhibit any toxic effect on neuronal cells at the concentrations at which this compound effectively prevents alfa-synuclein aggregation.

### REFERENCES

Benskey, M. J., Perez, R. G. and Manfredsson, F. P. (2016) The contribution of alpha synuclein to neuronal survival and function - Implications for Parkinson's disease', Journal of Neurochemistry, 137(3), pp. 331-359. doi: 10.1111/jnc.13570.

EP0676397 SHIONOGI & CO (2006) ‘EP0676397’.

Galvin, J. E., Lee, V. M.-Y. and Trojanowski, J. Q. (2001) ‘Clinical and Pathological Implications’, Arch Neurol, 58, pp. 186-190. doi: 10.1001/archneur.58.2.186.ABSTRACT.

Van Ham, T. J., Thijssen, K. L., Breitling, R., Hofstra, R. M. W., Plasterk, R. H. A. and Nollen, E. A. A. (2008) ‘C. elegans model identifies genetic modifiers of α-synuclein inclusion formation during aging’, PLoS Genetics, 4(3). doi: 10.1371/journal.pgen.1000027.

Herva, M. E., Zibaee, S., Fraser, G., Barker, R. A., Goedert, M. and Spillantini, M. G. (2014) ‘Anti-amyloid Compounds Inhibit??-Synuclein Aggregation Induced by Protein Misfolding Cyclic Amplification (PMCA)’, Journal of Biological Chemistry, 289(17), pp. 11897-11905. doi: 10.1074/jbc.M113.542340.

Howard, N. I., Dias, M. V. B., Peyrot, F., Chen, L., Schmidt, M. F., Blundell, T. L. and Abell, C. (2015) ‘Design and Structural Analysis of Aromatic Inhibitors of Type II Dehydroquinase from Mycobacterium tuberculosis’, ChemMedChem, 10(1), pp. 116-133. doi: 10.1002/cmdc.201402298.

Huttunen, K. M., Raunio, H. and Rautio, J. (2011) ‘Prodrugs—from serendipity to rational design.’, Pharmacological Reviews, 63(3), pp. 750-71. doi: 10.1124/pr.110.003459.enzymatic.

I.F., T., Y., S., V.L., K., J.P., G., W., W., C., O., T., G., M., T., B., S. and K., K. (2015) ‘Molecular determinants of alpha-synuclein mutants’ oligomerization and membrane interactions', ACS Chemical Neuroscience, 6(3), pp. 403-416. doi: 10.1021/cn500332w.

Miyaura, N. and Suzuki, A. (1995) ‘Palladium-Catalyzed Cross-Coupling Reactions of Organoboron Compounds’, Chemical Reviews, 95(7), pp. 2457-2483. doi: 10.1021/cr00039a007.

Moree, B., Yin, G., Lázaro, D. F., Munari, F., Strohäker, T., Giller, K., Becker, S., Outeiro, T. F., Zweckstetter, M. and Salafsky, J. (2015) ‘Small molecules detected by second-harmonic generation modulate the conformation of monomeric α-synuclein and reduce its aggregation in cells’, Journal of Biological Chemistry, 290(46), pp. 27582-27593. doi: 10.1074/jbc.M114.636027.

Muñoz-Lobato, F., Rodriguez-Palero, M. J., Naranjo-Galindo, F. J., Shephard, F., Gaffney, C. J., Szewczyk, N. J., Hamamichi, S., Caldwell, K. A., Caldwell, G. A., Link, C. D. and Miranda-Vizuete, A. (2014) ‘Protective Role of DNJ-27/ERdj5 in Caenorhabditis elegans Models of Human Neurodegenerative Diseases’, Antioxidants & Redox Signaling, 20(2), pp. 217-235. doi: 10.1089/ars.2012.5051.

Rautio, J., Kumpulainen, H., Heimbach, T., Oliyai, R., Oh, D., Jarvinen, T. and Savolainen, J. (2008) ‘Prodrugs: design and clinical applications.’, Pharmacological Reviews, 63(3), pp. 750-771. doi: 10.1124/pr.110.003459.enzymatic.

Reinartz, W., Krafft, M. and Hoyer, W. (2004) The customer relationship management process: its measurement and impact on performance', Journal of marketing research, 41(3), pp. 293-305. doi: 10.1509/jmkr.41.3.293.35991.

US2004052822 (KOHARA TOSHUYUKI ET AL) (2004) ‘United States (12)’, 1(19).

US2010041747 (FISCHER GUNTER ET AL.) (2010) ‘US2010041747’.

Watzky, M. A., Morris, A. M., Ross, E. D. and Finke, R. G. (2008) ‘Fitting yeast and mammalian prion aggregation kinetic data with the finke-watzky two-step model of nucleation and autocatalytic growth’, Biochemistry, 47(40), pp. 10790-10800. doi: 10.1021/bi800726m. Wermuth, C. (1996) The Practice of Medicinal Chemistry', The Practice of Medicinal Chemistry Academic Press, San Diego, pp. 671-715.

WO07110629 WISTA LABORATORIES LTD (2007) ‘WO07110629’.

WO2010015816 (SUMMIT CORP PLC) (2010) ‘wo10015816’.

WO2012080221 (UNIV LEUVEN KATH) (2012) WO2012080221A1′.

WO2014014937 NEUROPORE THERAPEUTICS INC (2014) ‘WO2014014937’.

1. A compound of formula I 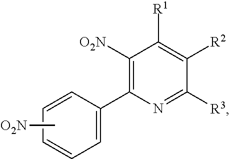
wherein
R1 is selected from C1-C4-alkyl or cyclopropyl, wherein up to three hydrogen atoms of the C1-C4-alkyl or of the cyclopropyl are optionally substituted by radicals which are independently selected from F, Cl, OH and NH2, provided there are no geminally bound OH groups if two or three OH groups are present,
R2 is selected from —CN, Cl and F, and
R3 is selected from OH, C1-C4-alkoxy and C1-C4-alkylcarbonyloxy, or a tautomer thereof, a pharmaceutically acceptable solvate thereof, a prodrug thereof, or pharmaceutically acceptable salt thereof,
provided that the compound is not a compound of formula II: 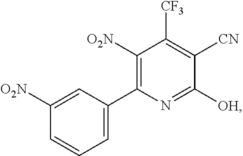 [SMILES: N#Cc1c(O)nc(-c2cccc([N+](=O)[O-])c2)c([N+](=O)[O-])c1C(F)(F)F]
formula IIb 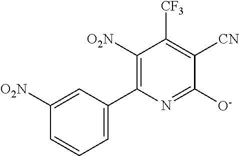 [SMILES: N#Cc1c([O-])nc(-c2cccc([N+](=O)[O-])c2)c([N+](=O)[O-])c1C(F)(F)F] 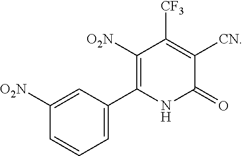 [SMILES: N#Cc1c(C(F)(F)F)c([N+](=O)[O-])c(-c2cccc([N+](=O)[O-])c2)[nH]c1=O]
or formula III

2. The pharmaceutical composition comprising a compound of formula I 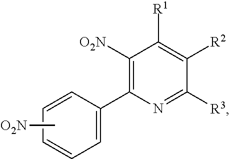
wherein
R1 is selected from C1-C4-alkyl or cyclopropyl, wherein up to three hydrogen atoms of the C1-C4-alkyl or of the cyclopropyl are optionally substituted by radicals which are independently selected from F, Cl, OH and NH2, provided there are no geminally bound OH groups if two or three OH groups are present,
R2 is selected from —CN, Cl and F, and
R3 is selected from OH, C1-C4-alkoxy and C1-C4-alkylcarbonyloxy, or a pharmaceutically acceptable solvate thereof, a prodrug thereof, or pharmaceutically acceptable salt thereof, and
at least one pharmaceutically acceptable carrier.
Provided the compound is not a compound of formula III

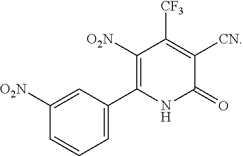 [SMILES: N#Cc1c(C(F)(F)F)c([N+](=O)[O-])c(-c2cccc([N+](=O)[O-])c2)[nH]c1=O]

3. The pharmaceutical composition of claim 2, wherein the compound of formula I is a compound of formula II 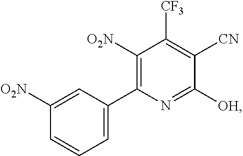 [SMILES: N#Cc1c(O)nc(-c2cccc([N+](=O)[O-])c2)c([N+](=O)[O-])c1C(F)(F)F]
or a pharmaceutically acceptable solvate thereof, a prodrug thereof, or pharmaceutically acceptable salt thereof.

4. The compound of formula I 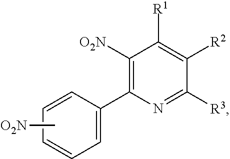
wherein
R1 is selected from C1-C4-alkyl or cyclopropyl, wherein up to three hydrogen atoms of the C1-C4-alkyl or of the cyclopropyl are optionally substituted by radicals which are independently selected from F, Cl, OH and NH2, provided there are no geminally bound OH groups if two or three OH groups are present,
R2 is selected from —CN, Cl and F, and
R3 is selected from OH, C1-C4-alkoxy and C1-C4-alkylcarbonyloxy,
or a pharmaceutically acceptable solvate thereof, a prodrug thereof, or pharmaceutically acceptable salt thereof,
for use in medicine.
Provided the compound is not a compound or formula III

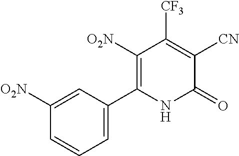 [SMILES: N#Cc1c(C(F)(F)F)c([N+](=O)[O-])c(-c2cccc([N+](=O)[O-])c2)[nH]c1=O]

5. The compound for use according to claim 4, wherein the compound is a compound of formula II 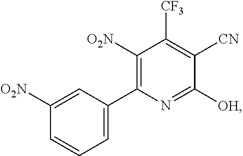 [SMILES: N#Cc1c(O)nc(-c2cccc([N+](=O)[O-])c2)c([N+](=O)[O-])c1C(F)(F)F]
or a pharmaceutically acceptable solvate thereof, a prodrug thereof, or pharmaceutically acceptable salt thereof.

6. The compound of formula I 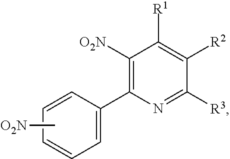
wherein
R1 is selected from C1-C4-alkyl or cyclopropyl, wherein up to three hydrogen atoms of the C1-C4-alkyl or of the cyclopropyl are optionally substituted by radicals which are independently selected from F, Cl, OH and NH2, provided there are no geminally bound OH groups if two or three OH groups are present,
R2 is selected from —CN, Cl and F, and
R3 is selected from OH, C1-C4-alkoxy and C1-C4-alkylcarbonyloxy,
or a tautomer thereof, a pharmaceutically acceptable solvate thereof, a prodrug thereof, or pharmaceutically acceptable salt thereof,
for use in the treatment or prophylaxis of a synucleinopathy.

7. The compound for use according to claim 6, wherein the compound is a compound of formula II 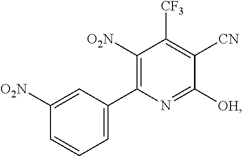 [SMILES: N#Cc1c(O)nc(-c2cccc([N+](=O)[O-])c2)c([N+](=O)[O-])c1C(F)(F)F]
or a tautomer thereof, a pharmaceutically acceptable solvate thereof, a prodrug thereof, or pharmaceutically acceptable salt thereof.

8. The compound for use according to claim 6, wherein the synucleinopathy is selected from Parkinson's Disease, Dementia with Lewy Bodies, Multiple System Atrophy, Pure Autonomic Failure, Lewy Body Variant of Alzheimer's Disease and Neurodegeneration with Brain Iron Accumulation.

9. The compound for use according to claim 6, wherein the synucleinopathy is familial.

10. The compound for use according to claim 6, wherein the subject of the treatment or the prophylaxis expresses α-synuclein comprising at least one amino acid substitution selected from:
a proline at position 30,
a lysine at position 46,
a glutamine at position 50,
an aspartate at position 51, and
a threonine or a glutamate at position 53;
the numbering of said amino acid positions being relative to the of full length α-synuclein as set forth in SEQ ID NO:1.

11. The compound for use according to claim 6, wherein the compound is for use in delaying the onset or the progression of the synucleinopathy.
